(19) United States
(12) Patent Application Publication
(10) Pub. No.: US 2023/0175052 A1
(43) Pub. Date: Jun. 8, 2023

(54) METHODS AND COMPOSITIONS FOR DETECTION OF NUCLEIC ACID SEQUENCE TARGETS

(71) Applicant: GALENVS SCIENCES INC., Montreal, Québec (CA)

(72) Inventor: Jamal DAOUD, Verdun, Québec (CA)

(21) Appl. No.: 17/913,346

(22) PCT Filed: Mar. 22, 2021
(86) PCT No.: PCT/CA2021/050378

§ 371 Date: Sep. 21, 2022

Related U.S. Application Data
(60) Provisional application No. 62/993,051, filed on Mar. 22, 2020.

(51) Int. Cl. C12Q 1/6855; B01L 3/00

(52) U.S. Cl. CPC C12Q 1/6855; B01L 3/502761; B01L 2300/0883; B01L 2200/0668

(57) ABSTRACT

The present disclosure provides, in various aspects and embodiments, methods, compositions, and devices for magnetic nanoparticle based assay of nucleic acid sequence targets using isothermal amplification. Uses of the disclosure include detection of bacterial and/or virus nucleic acid sequence targets.

### RELATED APPLICATIONS

This application claims the benefit of U.S. Provisional Application No. 62/993,051, filed Mar. 22, 2020, which is incorporated herein by reference in its entirety.

### BACKGROUND

The identification of nucleic acid sequence targets or fragments thereof for diagnostic purposes has long been a goal in the field of point-of-care (PoC) devices. Such an example can be applied to the discrimination between viral and bacterial infection for clinical and home diagnostic development. This is particularly important in epidemic/pandemic occurrences that call for rapid identification of infected individuals, such as the coronavirus disease 2019 (COVID-19) outbreak. Such a diagnostic tool could also prevent the over-prescription of antibiotics, a leading cause of antimicrobial resistance (AMR). Current standard methods involve sending patient samples (e.g., throat swabs, blood, urine) to specialized clinical labs. This usually involves protein biomarker assays for detection of pro-calcitonin and/or c-reactive protein (CRP). However, these techniques suffer from poor specificity and sensitivity in addition to patient variability. Furthermore, these classic methodologies do not provide a measure of antimicrobial susceptibility in the event of a bacterial infection.

This has led to the development of alternative detection and diagnostic methods for bacterial and viral infections through nucleic acid amplification tests (NAAT). While precise and sensitive, polymerase chain reaction (PCR)-associated methods are limited to clinical lab settings and very challenging to transfer to PoC diagnostic devices, given the need for thermal cycling and probe-based colorimetric or fluorescent detection. Such procedures necessitate the use of costly bench-top equipment, typically accompanied by disposable cartridges, in order to perform traditional thermal cycling and fluorescent-based quantification.

Thus, there is a need for more efficient methods to identify nucleic acid sequence targets or fragments thereof for diagnostic and PoC uses.

### SUMMARY OF THE DISCLOSURE

The present invention is directed to methods and compositions for detection of nucleic acid sequence targets and fragments thereof.

In one aspect, a method of immobilizing amplicons from a nucleic acid sequence target or fragment thereof is discussed, the method comprises mixing a fluid sample comprising the nucleic acid sequence target or fragment thereof, magnetic nanoparticles, primers, and an amplification reagent, wherein the magnetic nanoparticles comprise a first magnetic nanoparticle attached to a first probe and a second magnetic nanoparticle attached to a second probe, and wherein the first probe and the second probe are the same or different and optionally hybridize to different sections of the nucleic acid sequence target or the fragment thereof. The method comprises heating the fluid sample to activate the amplification reagent and amplify the nucleic acid sequence target or fragment thereof to form amplicons, thereby immobilizing the amplicons on the magnetic nanoparticles.

In some embodiments, the nucleic acid sequence target or fragment thereof can be a deoxyribonucleic acid (DNA) sequence, a ribonucleic acid (RNA) sequence, or a combination thereof. In some embodiments, the nucleic acid sequence target or fragment thereof can be a ribonucleic acid (RNA) sequence and the fluid sample further comprises a reverse transcriptase.

In some embodiments, the mixture can be heated at a temperature of at least about 30° C. or at least 50° C. In some embodiments, the mixture is heated at a temperature between about 60° C. and about 70° C. or between about 60° C. and about 65° C.

In some embodiments, the amplification reagent can be a Bst polymerase, a recombinase, a single-stranded DNA-binding protein, a strand-displacing polymerase, an enzyme designed for nucleic acid sequence-based amplification, an enzyme designed for helicase-dependent amplification, a nicking enzyme, any mutations thereof, or any combinations thereof. In some embodiments, the amplification reagent can yields amplicons with single-stranded regions, and the single-stranded regions can comprise single-stranded loop regions.

In some embodiments, the first probe and the second probe can hybridize to the single-stranded loop regions.

In some embodiments, the magnetic material comprises iron, cobalt, nickel, or any combination thereof. In some embodiments, the iron can be an iron oxide with a formula of Fe₃O₄, α-Fe₂O₃, β-Fe₂O₃ FeO, any derivatives thereof, or any combinations thereof. In some embodiments, the cobalt is a cobalt ferrite with a formula of CoFe, CoFe₂O₄, any derivatives thereof, or any combinations thereof.

In some embodiments, the magnetic nanoparticles can further comprise a linker connecting the probe to the surface of the magnetic material, wherein the linker is a functional group, a polymeric group, a dendritic group, or any combinations thereof. In some embodiments, the linker can be a functional group covalently coupling the probe to the surface of the magnetic material. In some embodiments, the covalent coupling comprises: a carboxyl-to-amine linkage using 1-ethyl-3-(3-dimethylaminopropyl) carbodiimide and N-hydroxy succinimide (NHS) or sulfo N-hydroxy succinimide; an amine-to-amine linkage using glutaraldehyde; an alkyne linkage using an alkyne click chemistry with cyanogen bromide; or any combinations thereof. In some embodiments, the linker can be a functional group comprising a hydroxyl, a carboxyl, an amine, a mercapto, an epoxy, an imidocarbonate, a cyanate ester, any derivates thereof, or any combinations thereof. In some embodiments, the linker can be a polymeric group comprising poly(ethylene glycol), poly(ethylenimine), poly(thioether), any derivatives thereof, or any combinations thereof. In some embodiments, the linker can be a dendritic group comprising poly(amidoamine), hyperbranched bis-MPA polyester-16-hydroxyl, any derivatives thereof, or any combinations thereof.

In some embodiments, the magnetic nanoparticles can have a diameter of at least about 10 nm or at least about 100 nm. In some embodiments, the magnetic nanoparticles can have a diameter between about 100 nm to about 1000 nm. In some embodiments, the magnetic nanoparticles comprising the immobilized amplicons can be agglutinates.

In some embodiments, the magnetic nanoparticles can further comprise a shell. In some embodiments, the thickness of the shell can be equal or less than about 5 nm. In some embodiments, the shell can comprise silica embedded with functional groups. In some embodiments, the functional groups can be hydroxyl, carboxyl, amine, mercapto, epoxy, imidocarbonate, cyanate ester, any derivatives thereof, or any combinations thereof.

In another aspect, a method for detecting a nucleic acid sequence target or fragment thereof in a fluid sample is discuss, the method comprises mixing the fluid sample comprising the nucleic acid sequence target or fragment thereof, magnetic nanoparticles, primers, and an amplification reagent, wherein the magnetic nanoparticles comprise a first magnetic nanoparticle attached to a first probe and a second magnetic nanoparticle attached to a second probe, wherein the first probe and the second probe are the same or different and optionally hybridize to different sections of the nucleic acid sequence target or the fragment thereof. The method comprises heating the fluid sample to activate the amplification reagent and amplify the nucleic acid sequence target or fragment thereof to form amplicons, thereby agglutinating the magnetic nanoparticles. The method comprises analyzing the agglutinates to detect the nucleic acid sequence target or fragment thereof.

In some embodiments, the method can further comprise the step of separating the agglutinates from the fluid sample before the analyzing step. In some embodiments, the separating step can comprise filtration, decantation, centrifugation, magnetism, or any combinations thereof. In some embodiments, the agglutinates can be analyzed by visual inspection. In some embodiments, the agglutinates can be analyzed by color intensity measurement, optical density measurement, or a combination thereof.

In some embodiments, the nucleic acid sequence target or fragment thereof can be a deoxyribonucleic acid (DNA) sequence, a ribonucleic acid (RNA) sequence, or a combination thereof. In some embodiments, the nucleic acid sequence target or fragment thereof is a ribonucleic acid (RNA) sequence and the fluid sample further comprises a reverse transcriptase.

In some embodiments, the mixture can be heated at a temperature of at least about 30° C. or at least 50° C. In some embodiments, the mixture can be heated at a temperature between about 60° C. and about 70° C. or between about 60° C. and about 65° C.

In some embodiments, the magnetic nanoparticles can have a magnetic moment of at least about 30 emu/g. In some embodiments the magnetic nanoparticles can have a magnetic moment between about 30 emu/g and about 200 emu/g. In some embodiments, the magnetic nanoparticles can be monodispersed. In some embodiments, the magnetic nanoparticles with the immobilized amplicons can have a magnetic moment of at least 100 emu/g.

In another aspect, a method of determining the amount of a nucleic acid sequence target or fragment thereof in a fluid sample is discussed, the method comprising mixing the fluid sample comprising the nucleic acid sequence target or fragment thereof, magnetic nanoparticles, and an amplification reagent, wherein the plurality of magnetic nanoparticles comprises a first magnetic nanoparticle attached to a first probe and a second magnetic nanoparticle attached to a second probe, wherein the first probe and the second probe are the same or different and optionally hybridize to different sections of the nucleic acid sequence target or the fragment thereof. The method comprises heating the fluid sample to activate the amplification reagent and amplify the nucleic acid sequence target or fragment thereof to form amplicons, thereby agglutinating the magnetic nanoparticles. The method comprises analyzing the agglutinates to determining the amount of nucleic acid sequence target or fragment thereof in the fluid sample.

In some embodiments, the method can further comprises the step of comparing the amount of nucleic acid sequence target or fragment thereof in the fluid sample to a threshold amount or to a control sample with a known amount of nucleic acid sequence target or fragment thereof.

In another aspect, a kit for detecting a nucleic acid sequence target or fragment thereof is discussed, the kit comprises a container suitable for mixing a fluid sample comprising the nucleic acid sequence target or fragment thereof, magnetic nanoparticles, and an amplification reagent, wherein the plurality of magnetic nanoparticles comprises a first magnetic nanoparticle attached to a first probe and a second magnetic nanoparticle attached to a second probe, wherein the first probe and the second probe are the same or different and optionally hybridize to different sections of the nucleic acid sequence target or the fragment thereof. The kit comprises a heater capable of heating the fluid sample and activating the amplification reagent to form amplicons, thereby agglutinating the magnetic nanoparticles. The kit comprises a means for analyzing the presence of, or the property of, the agglutinates, hereby detecting the nucleic acid sequence target or fragment thereof.

In another aspect, an apparatus for detecting a nucleic acid sequence target or fragment thereof using loop-mediated isothermal amplification is discussed, the apparatus comprises an entry valve for entering a fluid sample comprising the nucleic acid sequence target or fragment thereof, magnetic nanoparticles, and an amplification reagent, wherein the plurality of magnetic nanoparticles comprises a first magnetic nanoparticle attached to a first probe and a second magnetic nanoparticle attached to a second probe, wherein the first probe and the second probe are the same or different and optionally hybridize to different sections of the nucleic acid sequence target or the fragment thereof. The kit comprises a plurality of chambers fluidly connected to the entry valve and each other, wherein the chambers are designed to receive the fluid sample, wherein each of the chambers comprises a gel which is embedded with a primer that is specific to the nucleic acid target or fragment thereof. The kit comprises a heating device to heat the fluid sample, liquify the gel-laden primers in their respective chambers, and activate the amplification reagent to form amplicons by amplifying the nucleic acid sequence target or the fragment thereof using the probes hybridized to the nucleic acid sequence target or fragment, thereby agglutinating the magnetic nanoparticles. The kit comprises a magnet to interact with the agglutinates, thereby detecting the nucleic acid target or fragment thereof.

These aspects and embodiments, as well as other, are disclosed in further detail herein.

### BRIEF DESCRIPTION OF THE DRAWINGS

Aspects, features, benefits, and advantages of the embodiments described herein will be apparent with regard to the following description, appended claims, and accompanying drawings where:

FIG. 1 is a schematic illustration of an isothermal loop-mediated amplification (LAMP) amplicon with forward inner primer (FIP) and backward inner primer (BIP). The FIP primer comprises a F2 region at the 3′end and a F1c region at the 5′end. The F2 region is complementary to the F2c region of the template sequence. The F1c region is identical to the F1c region of the template sequence. The BIP primer comprises a B2 region at the 3′end and a B1c region at the 5′end. The B2 region is complementary to the B2c region of the template sequence. The B1c region is identical to the B1c region of the template sequence. Oligonucleotide probes targeting the loop region of LAMP amplicons (LB and LF probes) are covalently linked to magnetic nanoparticles (MNPs). As LAMP product amplicons are formed, the loop regions comprising single-stranded DNA are in turn hybridized to the MNPs decorated with LF and LB probes, in turn forming MNP agglutinates/aggregates.

FIG. 2 is a schematic illustration of a flocculation assay for detection of targets post LAMP.

FIG. 3 is a schematic illustration of a PoC diagnostic device for bacterial and/or viral targets or fragments thereof using magnetic bead LAMP and flocculation assay in multiple chambers with final visual detection of positive samples.

FIG. 4 is an image of a positive sample (E. Co/i) and a negative sample (negative) after a magnetic bead LAMP and flocculation assay for E. Co/i 0157 in Eppendorf tubes according to embodiments of the present teachings. Positive control samples which contain aggregated MNPs possess higher magnetic moment and in turn form a visually discernable ‘strip’, while negative control samples contain dispersed MNPs that remain as a stable colloidal dispersion.

FIG. 5 is an image of a microfluidic PoC platform containing lyophilized Bst polymerase, reverse transcriptase, BIP/FIP, F3/B3 primers as well as dNTPs and appropriate salts.

FIG. 6 is an image of a microfluidic PoC platform used to performed a LAMP assay to detect SARS-CoV-2 synthetic RNA. Positive control samples which contain aggregated MNPs possess higher magnetic moment and in turn form a visually discernable ‘strip’, while negative control samples contain dispersed MNPs that remain as a stable colloidal dispersion.

FIG. 7 is an image of an electrophoresis gel of an amplicon solution from a LAMP detection of SARS-CoV-2 synthetic RNA. This image further depicts the difference between a positive (+) and negative (−) LAMP reaction. Left lane: 1 kb ladder.

### DETAILED DESCRIPTION

It will be appreciated that for clarity, the following disclosure will describe various aspects of embodiments. It should be noted that the specific embodiments are not intended as an exhaustive description or as a limitation to the broader aspects discussed herein. The particular features, structures, or characteristics of the disclosed embodiments may be combined in any suitable manner, as would be apparent to a person skilled in the art from this disclosure.

### Definitions

Various terms are used herein consistent with their common meanings in the art. The following terms are defined below for clarity.

As used herein, the term “about” in quantitative terms refers to plus or minus 10% of the value it modifies (rounded up to the nearest whole number if the value is not sub-dividable, such as a number of molecules or nucleotides). For example, the phrase “about 100 mg” would encompass 90 mg to 110 mg, inclusive; the phrase “about 2500 mg” would encompass 2250 mg to 2750 mg. When applied to a percentage, the term “about” refers to plus or minus 10% relative to that percentage. For example, the phrase “about 20%” would encompass 18-22% and “about 80%” would encompass 72-88%, inclusive.

It is to be noted that the term “a” or “an” entity refers to one or more of that entity; for example, “a symptom,” is understood to represent one or more symptoms. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

It is understood that wherever aspects are described herein with the language “comprising,” otherwise analogous aspects described in terms of “consisting of” and/or “consisting essentially of” are also provided.

It will be appreciated that while a particular sequence of steps are shown and described herein for purposes of explanation, the sequence may be varied in certain respects, or the steps may be combined, while still obtaining the desired configuration. Additionally, modifications to the disclosed embodiment and the invention as claimed are possible and within the scope of this disclosed invention.

Disclosed herein are methods and compositions which apply isothermal amplification technology to detect nucleic acid targets in order to provide a rapid, facile and label-free method of nucleic acid target discrimination. The methods and compositions can be used without the need for benchtop instrumentation, thus enabling their use in a point-of-care (PoC) setting. As used herein, “nucleic acid target” or “nucleic acid sequence target” includes the nucleic acid sequence target as well as fragments thereof. The nucleic acid sequence target can be comprised of deoxyribonucleic acid (DNA) sequence, a ribonucleic acid (RNA) sequence, or a combination thereof.

Disclosed herein are methods and compositions which utilize functionalized magnetic nanoparticles (MNPs) and isothermal amplification to detect nucleic acid sequence targets of interest. Nucleic acid sequence targets of interest (e.g., bacterial, viral, gene targets) can be amplified in conjunction with the MNPs functionalized with probes (e.g., poly probes, oligo probes) against the nucleic acid sequence targets or fragments thereof.

Using the methods and compositions disclosed herein, the functionalized MNPs become increasingly incorporated with the isothermal amplification reaction, resulting in the immobilization of amplicons and agglutination of the MNPs. The agglutinated MNPs have an increased magnetic moment as compared to the MNPs which remain dispersed.

Consequently, the agglutinated MNPs in the presence of a magnetic field will form a tunable pattern that can be visually apparent. A positive amplification event of the nucleic acid sequence targets or fragments thereof can therefore be assessed visually without any label. This provides a significant advantage over other nucleic acid amplification test (NAAT)-based diagnostics which rely on quantitative PCR and traditional thermal cycling and fluorescent-based detection.

In some embodiments, the agglutination of the MNPs can be analyzed using a flocculation assay. This flocculation assay can be used to readily identify the presence, absence, or quantity of a specific nucleic acid sequence target according to the methods and compositions disclosed herein.

In some embodiments, the methods and compositions disclosed herein are used to discriminate between bacterial and viral infection.

### Isothermal Amplification

The methods and compositions disclosed herein apply isothermal amplification to immobilize amplicons from a nucleic acid sequence target or fragment thereof. The isothermal amplification techniques for the assaying of the nucleic acid sequence targets or fragments thereof disclosed herein can be any isothermal amplification technique that is known in the art that yields amplicons with single stranded loop regions and/or other single stranded amplicons, for example asymmetric PCR. These isothermal amplification techniques include for example, without limitation, loop-mediated isothermal amplification (LAMP), rolling circle amplification (RCA), recombinase polymerase activation (RPA), nucleic acid sequence-based amplification (NASBA), helicase-dependent amplification (HDA), nicking enzyme amplification reaction (NEAR), or any combinations thereof.

Conventional asymmetric PCR using limiting and/or excess primers can also be used as a source of single stranded DNA (ssDNA) amplicons. Resulting single strands may also be used as a template for probe-functionalized MNPs specific for amplified ssDNA. These asymmetric PCR techniques include Linear-After-The-Exponential (LATE)-PCR non-enzymatic amplification techniques.

Non-enzymatic amplification techniques such as without limitation Ribozymes, DNAzymes, and toehold mediated strand displacement (TMSD), or general strand displacement reactions (SDR), may also be used to generate single stranded products. Such generated single stranded oligonucleotide, ssDNA and/or RNA output may also be used as template for probe-functionalize MNPs.

The LAMP isothermal amplification method amplifies nucleic acid sequences with high specificity, efficiency and rapidity under isothermal conditions. This method employs a DNA polymerase and a set of primers that recognize a distinct sequences on the target DNA. The method can employ a set of about 4 or more primers, about 6 or more primers, about 8 or more primers, and about 10 or more primers. Generally, an inner primer containing sequences of the sense and antisense strands of the target DNA initiates LAMP. The following strand displacement DNA synthesis primed by an outer primer releases a single-stranded DNA. This serves as template for DNA synthesis primed by the second inner and outer primers that hybridize to the other end of the target, which produces a stem-loop DNA structure. In subsequent LAMP cycling one inner primer hybridizes to the loop on the product and initiates displacement DNA synthesis, yielding the original stem-loop DNA and a new stem-loop DNA with a stem twice as long. The cycling reaction continues with accumulation a large number of copies of target in a short period of time. The final products are stem-loop DNAs with several inverted repeats of the target and cauliflower-like structures with multiple loops formed by annealing between alternately inverted repeats of the target in the same strand. Because LAMP recognizes the target by six distinct sequences initially and by four distinct sequences afterwards, it is expected to amplify the target sequence with high selectivity. Design and selection of appropriate primers for LAMP amplification is well known to one of skill in the art.

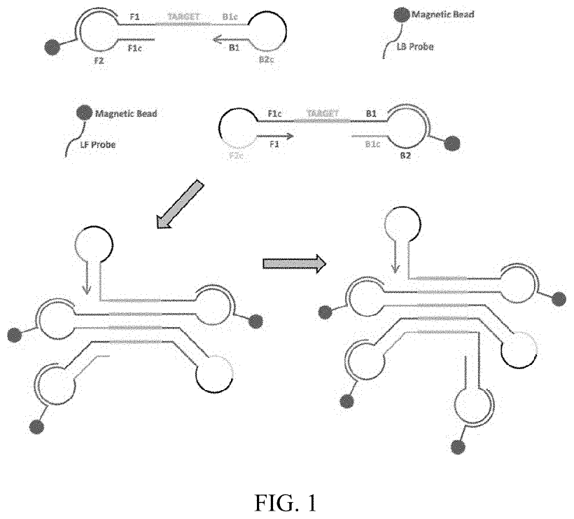

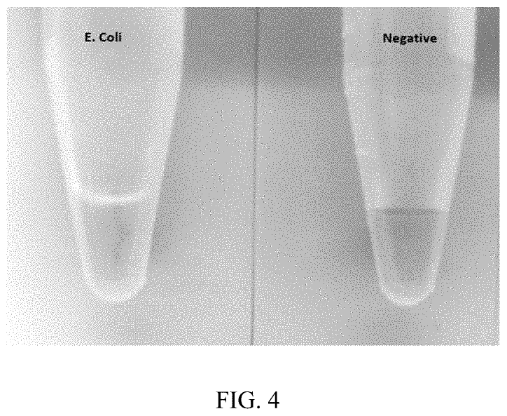

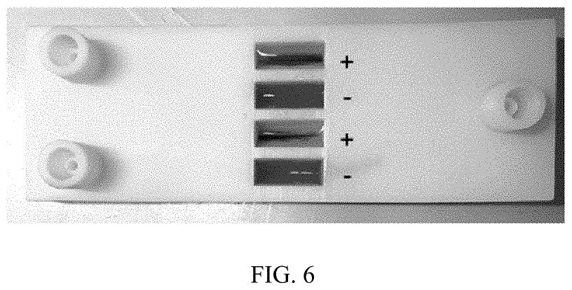

FIG. 1 schematically illustrates a magnetic nanoparticle based assay of nucleic acid sequence targets or fragments thereof using the isothermal amplification technique LAMP. The LAMP assay is performed to amplify the nucleic acid sequence targets or fragments of interest in conjunction with MNPs functionalized with oligo probes against the target sequence. Without being bound by theory, as amplification of the specific nucleic acid sequence target occurs, the MNPs become increasingly incorporated with the LAMP reaction, and the number and size of MNP agglutinates will increase. When subjected to a magnetic field, the MNPs will align with the magnetic field to form a visually discernable signal. For example, in the flocculation assay disclosed herein, a positive signal is indicated by a distinct strip or precipitate (see, e.g. FIG. 4 and FIG. 6). In the absence of the nucleic acid sequence target, there is minimal MNP agglutination. As such, MNPs remain dispersed and do not form the visually discernable signal (e.g., the distinct strip or precipitate) when subjected to a magnetic field, indicating a negative signal.

The NASBA isothermal amplification method is used to amplify RNA. The HDA isothermal amplification method uses the double-stranded DNA unwinding activity of a helicase to separate strands for DNA amplification at constant temperature. The RCA isothermal amplification method starts with a circular DNA template and a short DNA or RNA primer to form a long single stranded molecule. The MDA isothermal amplification method is a technique that initiates when multiple random primers anneal to a DNA template and a polymerase amplifies DNA at constant temperature. The RPA isothermal amplification method is a low temperature DNA and RNA amplification technique.

The RPA isothermal amplification method is a process that generally employs three core enzymes: a recombinase, a single-stranded DNA-binding protein (SSB) and strand-displacing polymerase. Recombinases are capable of pairing oligonucleotide primers with homologous sequence in duplex DNA. SSB binds to displaced strands of DNA and prevent the primers from being displaced. Finally, the strand displacing polymerase begins DNA synthesis where the primer has bound to the target DNA. By using two opposing primers, much like PCR, if the target sequence is indeed present, an exponential DNA amplification reaction is initiated. There is no other sample manipulation such as thermal or chemical melting is required to initiate amplification.

The isothermal amplification assays disclosed herein are performed at a constant temperature. Examples of temperatures for the isothermal amplification assays include, for example, at least about 30° C., a least about 32° C., a least about 34° C., a least about 36° C., a least about 38° C., a least about 40° C., a least about 42° C., a least about 44° C., a least about 46° C., a least about 48° C., a least about 50° C., a least about 52° C., a least about 54° C., a least about 56° C., a least about 58° C., a least about 60° C., or any ranges that are made of any two or more points in the above list. In some embodiments, the isothermal amplification assay temperature is between about 30° C. and about 50° C., between about 35° C. and about 45° C., or between about 37° C. and about 42° C.

The isothermal amplification assays disclosed herein can progress rapidly and result in the amplification of specific nucleic acid sequence targets or fragments thereof from just a few target and fragment copies to a detectable level target and fragment copies. This process can be performed within about 5 minutes, within about 10 minutes, within about 15 minutes, within about 20 minutes, within about 25 minutes, within about 30 minutes, within about 35 minutes, within about 40 minutes, within about 45 minutes, within about 50 minutes, within about 55 minutes, within about 60 minutes, within about 65 minutes, within about 70 minutes, within about 75 minutes, within about 80 minutes, within about 85 minutes, within about 90 minutes, within about 95 minutes, within about 100 minutes, within about 105 minutes, within about 110 minutes, within about 115 minutes, within about 120 minutes, or within any ranges that are made of any two or more points in the above list.

The isothermal amplification assays disclosed herein comprises one or more amplification reagents. Examples of amplification reagents include, without limitation, Bst polymerase, a recombinase, a single-stranded DNA-binding protein, a strand-displacing polymerase, an enzyme designed for nucleic acid sequence-based amplification, an enzyme designed for helicase-dependent amplification, a nicking enzyme, or any combinations thereof. Derivatives or mutations of amplification reagents can also be used as required for the specific isothermal amplification.

### Magnetic Nanoparticles (MNPs)

The methods and compositions disclosed herein couple isothermal amplification with functionalized magnetic nanoparticles (MNPs).

In some embodiments, the MNPs useful in the methods and compositions disclosed herein are superparamagnetic nanoparticles. Before isothermal amplification, the MNP disclosed herein are monodispersed in a stable colloidal suspension. In some embodiments, the dispersed MNPs have a magnetic moment of at least about 30 emu/g, at least about 35 emu/g, at least about 40 emu/g, at least about 45 emu/g, at least about 50 emu/g, at least 55 emu/g, at least 60 emu/g, at least 65 emu/g, at least 70 emu/g, at least 75 emu/g, at least about 80 emu/g, about 85 emu/g, about 90 emu/g, about 95 emu/g, at least about 100 emu/g, at least about 105 emu/g, at least about 120 emu/g, at least about 125 emu/g, at least about 130 emu/g, at least about 135 emu/g, at least about 140 emu/g, at least about 145 emu/g, at least about 150 emu/g, at least about 155 emu/g, at least about 160 emu/g, at least about 165 emu/g, at least about 170 emu/g, at least about 175 emu/g, at least about 180 emu/g, at least about 185 emu/g, at least about 190 emu/g, at least about 195 emu/g, at least about 200 emu/g, or within any ranges that are made of any two or more points in the above list.

For use in the flocculation assay described herein, MNPs useful for the methods disclosed herein must form MNP agglutinates/aggregates with a higher magnetic moment than the dispersed MNPs. In some embodiments, the agglutinated/aggregated MNPs have a magnetic moment of at least about 80 emu/g, about 85 emu/g, about 90 emu/g, about 95 emu/g, at least about 100 emu/g, at least about 105 emu/g, at least about 120 emu/g, at least about 125 emu/g, at least about 130 emu/g, at least about 135 emu/g, at least about 140 emu/g, at least about 145 emu/g, at least about 150 emu/g, at least about 155 emu/g, at least about 160 emu/g, at least about 165 emu/g, at least about 170 emu/g, at least about 175 emu/g, at least about 180 emu/g, at least about 185 emu/g, at least about 190 emu/g, at least about 195 emu/g, at least about 200 emu/g, at least about 205 emu/g, at least about 210 emu/g, at least about 215 emu/g, at least about 220 emu/g, at least about 225 emu/g, at least about 230 emu/g, at least about 235 emu/g, at least about 240 emu/g, at least about 245 emu/g, at least about 250 emu/g, or within any ranges that are made of any two or more points in the above list.

Probe-functionalized MNPs can target any single stranded region within a PCR amplicon such as without limitation the loop region of LAMP products. Due to the presence of multiple complementary sites, an MNP agglutinate/aggregate is formed containing many MNPs hybridized with single or multiple amplicons.

MNP agglutinates/aggregates formed using the methods and compositions disclosed herein can be analyzed using visual inspection. This represents a significant advantage as compared to other techniques for detection of nucleic acid sequence targets. However, analysis of the MNP agglutinates/aggregates can be performed using a broad range of different techniques. In some embodiments, the MNP agglutinates/aggregates are analyzed by color intensity measurement, optical density measurement, nuclear magnetic resonance spectroscopy measurement, or any combinations of measurement thereof.

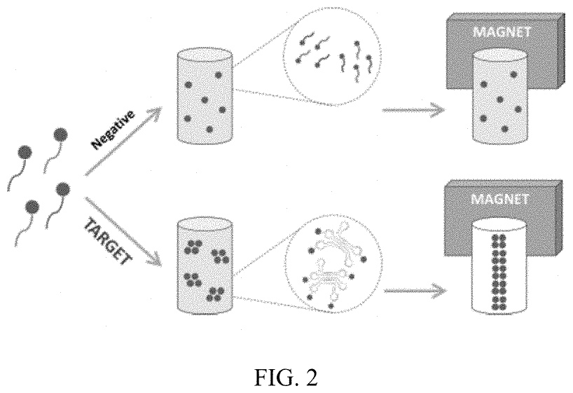

In a specific embodiment, analysis of the MNP agglutinates/aggregates is performed using a flocculation assay. A schematic of the flocculation assay disclosed herein is illustrated schematically in FIG. 2.

To obtain visually distinct flocculation during magnetic capture, several parameters can be readily optimized, including: (i) magnetic bead composition and size; (ii) external magnet strength; and (iii) buffer formulation. Machine learning approaches for biochemical process optimization may also be applied to improve specific applications of the disclosed methods.

MNPs useful in the methods disclosed herein can exhibit superparamagnetic behavior and be modified with appropriate functional groups. In certain embodiments, the MNPs further comprise a shell. In some embodiments, the shell comprises silica embedded with functional groups. To ensure access of functional groups, the shell thickness of the MNPs does not exceed about 5 nm. In some embodiments, MNPs functionalized with hydroxyl (—OH), carboxyl (—COOH), amine (—NH₂), mercapto (—SH), epoxy, imidocarbonate, cyanate ester, any derivatives thereof, or any combinations thereof are activated to allow for covalent coupling of a probe and/or linker. Non-limiting examples of MNPs useful in the methods and compositions disclosed herein are surface-activated Dynabeads (ThermoFisher) and magnetic microspheres (Bangs Laboratories Inc.).

MNPs comprise magnetic materials which include without limitation, magnetic metals such as cobalt, nickel, iron, and mixtures thereof, ferromagnetic alloys of magnetic metals, and magnetic iron oxides such as magnetite, maghemite, and ferrites. In some embodiments, the magnetic material is an iron oxide, including for example Fe₃O₄, α-Fe₂O₃, β-Fe₂O₃, FeO, any derivatives thereof, or any combinations thereof. In some embodiments, the magnetic material is a cobalt ferrite, including for example CoFe, CoFe₂O₄, any derivatives thereof, or any combinations thereof. In some embodiments, the MNPs further comprise silane reactive polymers.

The magnetic nanoparticles can have a diameter of at least about 5 nm, at least about 10 nm, at least about 20 nm, at least about 30 nm, at least about 40 nm, at least about 50 nm, at least about 60 nm, at least about 70 nm, at least about 80 nm, at least about 90 nm, at least about 100 nm, about 110 nm, at least about 120 nm, at least about 130 nm, at least about 140 nm, at least about 150 nm, at least about 160 nm, at least about 170 nm, at least about 180 nm, at least about 190 nm, at least about 200 nm, about 210 nm, at least about 220 nm, at least about 230 nm, at least about 240 nm, at least about 250 nm, at least about 260 nm, at least about 270 nm, at least about 280 nm, at least about 290 nm, at least about 300 nm, about 310 nm, at least about 320 nm, at least about 330 nm, at least about 340 nm, at least about 350 nm, at least about 360 nm, at least about 370 nm, at least about 380 nm, at least about 390 nm, at least about 400 nm, about 410 nm, at least about 420 nm, at least about 430 nm, at least about 440 nm, at least about 450 nm, at least about 460 nm, at least about 470 nm, at least about 480 nm, at least about 490 nm, at least about 500 nm, about 510 nm, at least about 520 nm, at least about 530 nm, at least about 540 nm, at least about 550 nm, at least about 560 nm, at least about 570 nm, at least about 580 nm, at least about 590 nm, at least about 600 nm, about 610 nm, at least about 620 nm, at least about 630 nm, at least about 640 nm, at least about 650 nm, at least about 660 nm, at least about 670 nm, at least about 680 nm, at least about 690 nm, at least about 700 nm, about 710 nm, at least about 720 nm, at least about 730 nm, at least about 740 nm, at least about 750 nm, at least about 760 nm, at least about 770 nm, at least about 780 nm, at least about 790 nm, at least about 800 nm, about 810 nm, at least about 820 nm, at least about 830 nm, at least about 840 nm, at least about 850 nm, at least about 860 nm, at least about 870 nm, at least about 880 nm, at least about 890 nm, at least about 900 nm, about 910 nm, at least about 920 nm, at least about 930 nm, at least about 940 nm, at least about 950 nm, at least about 960 nm, at least about 970 nm, at least about 980 nm, at least about 990 nm, at least about 1,000 nm, or any ranges that are made of any two or more points in the above list.

The functionalized MNPs disclosed herein further comprise a probe which specifically interacts with the nucleic acid sequence target or fragment thereof. In some embodiments, the functionalized MNPs comprise a first MNP attached to a first probe (e.g. LF probe) and a second MNP attached to a second probe (e.g. LB probe), wherein the first MNP and the second MNP are the same or different, and wherein the first probe and the second probe are the same or different and optionally hybridize to different sections of the nucleic acid sequence target or fragment thereof. In some embodiments, the probes are oligonucleotides, polynucleotides, or a combination thereof. Oligonucleotide and polynucleotide probes can be modified with a reactive functional group at the 5′ or 3′ end. The LF and LB probes can be designed as a complementary sequence to the single stranded loop region of LAMP amplicons. The probes are similar in sequence to the LF and LB primers used in conventional LAMP reactions, and can be used in lieu of these primers, either replacing a single primer (LB or LF probe for LB or LF primers, respectively) or both primers (LB/LF probes in place of LB/LF primers). In some embodiments, the oligonucleotide and/or polynucleotide probes are designed to target a single-stranded PCR amplicon. In certain embodiments, the disclosed oligonucleotide and/or polynucleotide probes are designed to target the loop region of LAMP amplicons. Design and generation of oligonucleotide and polynucleotide probes targeting these amplicons are known in the art. Other probes that can be used include without limitation antibodies and antigens.

The probe can be connected to the surface of the MNP by a linker. The linker can be covalently bonded to the MNP and/or the probe. The linker can be a functional group, a monomeric group, a polymeric group, any derivatives thereof, and any combinations thereof. The linker can be linear, branched, dendritic, any derivatives thereof, and any combinations thereof. Examples of linkers include, without limitation, coupling reagents such as 1′-carbonyl diimidazole (CDI), N,N′-dicyclo hexylcarbodiimide (DCC), (3-Dimethylaminopropyl)-N′-ethyl carbodiimide hydrochloride (EDC or EDCI), and derivatives thereof, polymers such as poly poly(ethylene glycol), poly(ethylenimine), poly(thioether), and any derivatives thereof, functional groups such hydroxy group, amino group, mercapto group, epoxy group, imidocarbonate group, cyanate ester group, and any derivatives thereof, and dendrimer such as poly(amidoamine), hyperbranched bis-MPA polyester-16-hydroxyl, and any derivatives thereof. Covalent coupling of the linker can be performed in several ways, including carboxyl-to-amine crosslinking the carbodiimide 1-Ethyl-3-(3-dimethylaminopropyl)carbodiimide (EDC) and N-hydroxy succinimide (NHS), or Sulfo-NHS coupling, amine-amine linkage using glutaraldehyde as a crosslinking agent, and/or alkyne-based click chemistry with cyanogen bromide.

### Test Samples

One skilled in the art will appreciate that the isothermal amplification assays disclosed herein can be used to test any type of fluid sample. Examples of fluid samples that can be used with the isothermal amplification assays include, without limitation, serological fluid sample including body fluid, serum, blood, saliva, mucus such as nasal mucus and throat mucus, tears, milk, urine, any derivatives thereof, or any combinations thereof. The fluid sample can comprise one or more liquids that have been used to dilute the fluid sample and/or to rinse one or more solids. Examples of other fluids include, without limitation, water such as tap water, distilled water, milli-Q water, and electrolyte water, buffer, eluent, any combinations thereof, and any derivatives thereof. Examples of solids include, without limitation, swab, cells, tissues, and surface such as medical equipment surface. In some embodiments, the amount of liquid associated with the fluid sample is sufficient to transfer the fluid sample on the one or more entry zones of a point-of-care device.

In some embodiments, the fluid sample is combined with a sample preparation buffer. The sample preparation buffer can be optimized to: (i) maximize the isothermal amplification assay products and the incorporation of the MNPs into the assay products, and (ii) ensure efficient extraction of nucleic acid sequence target through lysis of the organism (e.g. bacteria or virus). The sample preparation buffer can also be lyophilized.

The fluid sample can be transferred to a point-of-care (PoC) device using a collection device. The collection device can be any collection devices that can collect a fluid sample. Examples of collection device include, without limitation, swab, pipette, tube such as capillary tube, bag, and any products thereof.

In some embodiments, a serological fluid sample is non-invasively collected with the collection device. The collection step can include wiping or dabbing the collection device over a surface of a body containing the serological fluid sample to be tested.

In some embodiments, a serological fluid sample, such as blood, is collected in a pipette or other collection device. The collection step can include obtaining blood, for example using a lancet or a finger prick, and collecting it with a pipette or other collection device.

In some embodiments, no collection device is needed to transfer the fluid sample to the diagnostic device. For example, without limitation, a blood sample can be obtain from a finger prick and directly deposited on the one or more entry zones of a point-of-care device.

In some embodiments, the fluid sample can be used without any pre-treatment. For example, a blood droplet could be directly placed in the entry zone of a point-of-care device.

In some embodiments, the fluid sample can be used with pre-treatment. The fluid sample can be pretreated with one or more stabilization agents. Examples of stabilization agents include, without limitation, a sugar (e.g. sucrose), surfactant, polyol, and/or buffer.

The collection device can be dry or wet, sterile or not sterile, pretreated or not pretreated, or any combinations thereof before the collection step. In some embodiments, the collection device is treated with one or more blocking agents, one or more stabilization agents, or any combinations thereof. Examples of blocking agents include, without limitation, BSA and casein.

In some embodiments, the fluid sample and/or the collection device are not pretreated. By not subjecting the fluid sample and/or the collection device to pre-treatment, degradation of the fluid sample may be avoided. Without wishing to be bound to any particular theory, it is believed that the analytes to be tested can remain intact or in their native form surrounded or mixed with the other naturally occurring substances present in the fluid sample.

One skilled in the art will appreciate that the isothermal amplification assays disclosed herein can be used to test nucleic acid sequence targets or fragments thereof from any origin such human, animal, plants, virus, bacterial, alga, fungus, and yeast. An example of virus is SARS coronavirus such as the SARS-CoV-2 (COVID-19) virus. Other examples of virus include, without limitation, Yellow fever virus, Zika virus, Adeno-associated virus, Influenza virus, Aichi virus, Papillomavirus, Parainfluenza, Rubella virus, BK polyomavirus, Banna virus, Bunyamwera virus, Bunyavirus virus, Human Immunodeficiency Virus, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebola virus, Echovirus, Encephalomyocarditis virus, Epstein-Barr virus, European bat lyssaviru, Hepatitis virus, Horsepox virus, Adenovirus, Astrovirus, Cytomegalovirus, Enterovirus, Herpesvirus, Immunodeficiency virus, Parvovirus, Respiratory syncytial virus, Rhinovirus, Spumaretrovirus, T-lymphotropic virus, Torovirus, Isfahan virus, JC polyomavirus, Japanese encephalitis virus, Junin arenavirus, KI Polyomavirus, Kunjin virus, Lagos bat virus, Langat virus, Louping ill virus, Lymphocytic choriomeningitis virus, MERS coronavirus, Measles virus, Mengo encephalomyocarditis virus, Merkel cell polyomavirus, Molluscum contagiosum virus, Monkeypox virus, Mumps virus, Rabies virus, Rosavirus, Rotavirus, Sagiyama virus, Salivirus, Sandfly fever sicilian virus, Simian virus, Sindbis virus, Tick-borne powassan virus, Varicella-zoster virus, Variola virus, Vesicular stomatitis virus, West Nile virus, and any mutations thereof.

### Point-of-Care Diagnostic Device

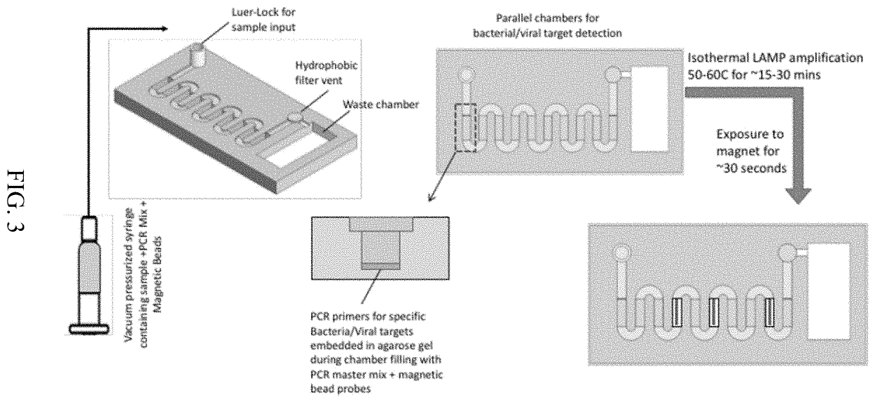

The methods and compositions disclosed herein can be applied using a point-of-care (PoC) device. The methods and compositions disclosed herein can be applied to create a cost-effective, rapid, user-friendly, and/or field deployable PoC solution for target nucleic acid detection, e.g., nucleic acid amplification test (NAAT). The unique magnetic behavior of the MNPs during the isothermal amplification enables the PoC device user to identify the presence or absence of nucleic acid sequence targets or fragments thereof in a rapid flocculation assay format. A schematic of an exemplary PoC device according to the instant disclosure is provided in FIG. 3.

The PoC diagnostic device disclosed herein may be disposable and/or made of plastic. The PoC diagnostic device can encompass multiple serially and fluidly connected chambers to perform isothermal amplification assays. The chambers may contain immobilized primers for the isothermal amplification which are laden in thermo-sensitive hydrogels such as gelatin or agarose, or other thermally sensitive materials such as parafilm, which liquify during isothermal amplification. The device can also be equipped with a vented waste chamber as to prevent contamination of potentially biohazardous waste.

The PoC diagnostic device can be coupled to a filling and/or sample collection module device such as a syringe. The syringe can be filled with a fluid sample comprising functionalized MNPs and the necessary reagents for the isothermal amplification, such as salts and enzymes. The PoC device can be readily stored with a long shelf life, as the MNPs used in the disclosed methods and compositions are extremely stable when dispersed in a colloidal solution. In some embodiments, the syringe contains a fluid sample comprising the nucleic acid sequence target or fragment thereof, magnetic nanoparticles (MNPs), primers, and an amplification reagent.

An external magnetic field may be applied following the isothermal amplification assay—whereby a heating source such as thin film resistors or heaters can be employed—in order to perform the flocculation assay with functionalized MNPs for visual detection of positive reactions.

### EXAMPLES

The following examples are provided merely to illustrate various embodiments of the invention, and are not intended to be limiting in any way.

### Example 1: Probe Functionalization of Magnetic Nanoparticles

Magnetic nanoparticles (MNPs) according to the methods and compositions disclosed herein were functionalized with probes using the following procedure:

Magnetite iron oxide MNPs were synthesized using a thermal decomposition method to obtain a monodisperse size of 10 nm, exhibiting superparamagnetic behavior. 15 mg of MNPs were capped with citric acid to obtain a stable colloidal dispersion and surface functional carboxyl groups. Oligonucleotide probes (loop backward, LB, and loop forward, LF) targeting the single-stranded loop regions of LAMP amplicons were designed and commercially ordered containing a 5′ amino functional group. MNPs containing carboxyl functionality were activated using 80 mM carbodiimide 1-Ethyl-3-(3-dimethylaminopropyl)carbodiimide (EDC) and 20 mM N-hydroxy succinimide (NHS) suspended in 50 mM 2-(N-morpholino)ethanesulfonic acid (MES) buffer and 0.01% Triton X at pH 5.5. After 20 minutes, MNPs were washed twice with MES buffer pH 5.5 using amicon spin column filtration devices with 100 kDa cut-off. MNPs were then resuspended in separate solutions of phosphate buffered saline (PBS) at pH 7.4 and 0.01% Triton X—one containing 10 nM concentration of LB probe and the other 10 nM LF probe. Following overnight incubation with agitation, the MNPs were washed twice with Tris-HCl buffer pH 7 to quench unreacted carbodiimide esters using amicon spin column filtration devices with 100 kDa cut-off. Finally, LB-MNPs and LF-MNPs were then suspended in ultrapure water at concentration of 15 mg/mL and stored at 4° C. until use.

### Example 2: Amplification and Detection of Bacterial DNA Using MNP-LAMP and Flocculation Assay

E. coli 0157 genomic DNA was detected in a test sample using the methods and compositions disclosed herein.

Briefly, the test sample was introduced to a microfuge tube containing Bst 2.0 polymerase, reverse transcriptase (RT), backward inner primer (BIP)/forward inner primer (FIP), forward outer primer (F3)/backward outer primer (B3), and loop forward primer (LF), as well as dNTPs and appropriate salts for LAMP. Primer were selected specifically for the rfbE gene of E. Coli 0157, adapted from Wang et al., 2012 (J Clin Microbiol. 2012 January; 50(1): 91-9). LAMP buffer was prepared containing the following components: 20 mM Tris-HCl (pH=8.8); 10 mM KCl; 10 mM (NH₄)₂SO₄; 8 mM MgSO4; 1.4 mM each dNTP; 7.5 U RTx Reverse transcriptase; 8 U Bst DNA polymerase; 40 pmol of FIP and BIP; 20 pmol of LF; and 5 pmol of F3 and B3. A positive sample of 100 copies of E. Co/i 0157 genomic DNA was prepared (Sigma-Aldrich, St. Louis, Mo., USA, product #: IRMM449). The E. Co/i 0157 genomic DNA was diluted to appropriate concentration in ultrapure water and in turn introduced to the microfuge tube containing the LAMP buffer. The LAMP buffer was prepared such that the final reaction volume was 100 μL, containing 4 μL of target DNA. A negative sample was also prepared in a separate microfuge tube, containing the same LAMP buffer but no target. LB functionalized MNPs were introduced to the LAMP buffer in lieu of the LB primer at a final concentration of about 500 μg/mL in the LAMP buffer by introducing 5 μL of 10 mg/mL into the reaction solution for a final volume of 100 μL. The tubes were placed on a heating block at 65° C. for 20 minutes. The subsequent flocculation assay is depicted in FIG. 4. The positive result is demonstrated by a ‘strip’ pattern.

### Example 3: Amplification and Detection of SARS-CoV-2 RNA Using MNP-LAMP and Flocculation Assay

The methods and compositions disclosed herein were used to detect SARS-CoV-2 RNA in a test sample.

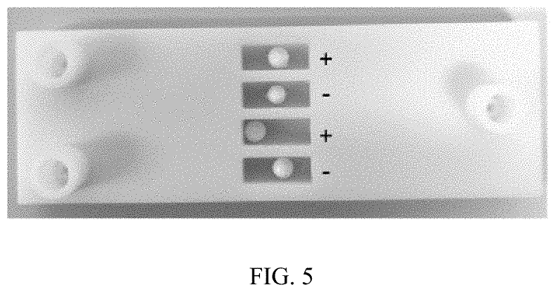

Briefly, the test sample was used with a microfluidic PoC platform containing lyophilized Bst 2.0 polymerase, reverse transcriptase (RT), BIP/FIP, F3/B3, and LF primers, as well as dNTPs and appropriate salts for LAMP (FIG. 5). The PoC platform was 3D printed to contain 4 parallel reaction chambers measuring 5 mm×2 mm×2 mm. LAMP buffer was prepared and lyophilized into 2 mm pellets containing the following components: 20 mM Tris-HCl (pH=8.8); 10 mM KCl; 10 mM (NH₄)₂SO₄; 8 mM MgSO₄; 1.4 mM each dNTP; 7.5 U RTx Reverse transcriptase; 8 U Bst DNA polymerase; 40 pmol of FIP and BIP; 20 pmol of LF; and 5 pmol of F3 and B3. Lyophilized pellets were formulated for resuspension with 100 μL of ultrapure water containing LB-functionalized MNPs at a concentration of about 500 μg/mL in order to obtain appropriate LAMP component concentrations, as described further below. 2 pellets were also supplemented with synthetic RNA for SARS-CoV-2. Synthetic RNA was introduced at a final concentration of 100 copies/4 from a stock solution of 10 e⁶ copies/4, procured from Twist Biosciences (San Francisco, Calif., USA).

The lyophilized pellets were introduced into the PoC chambers and sealed with transparent PCR sealer (FIG. 6). LB and LF functionalized MNPs were prepared as described in Example 1. Primer and adapted probe sequences were selected specifically for SARS-CoV-2 N gene and adapted from Ganguli et al., 2020 (Proc Natl Acad Sci USA. 2020 Sep. 15; 117(37):22727-22735).

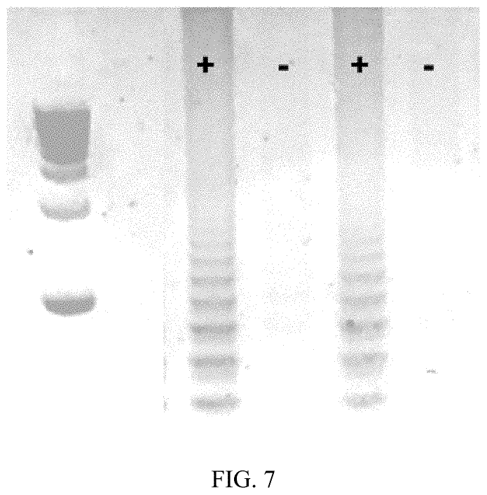

The MNP solution was diluted to a concentration of 500 μg/mL and introduced to the chambers. The lyophilized pellets were then reconstituted with the MNP solutions—100 uL of ultrapure water containing the functionalized MNPs—and the PoC platform was placed on a heating block at 65° C. for 20 minutes. The PoC platform was then removed and placed on top of a magnetic plate with pull force of 20 lbs. After 5 minutes, the chambers with the positive targets was amplified and form an MP assembly possessing a high magnetic moment, which results in MP aggregates forming a ‘strip’ pattern. The chambers with negative targets did not form LAMP amplicons and MPs remain dispersed in solution. A gel electrophoresis image of the amplicon solution also showed the difference between positive and negative LAMP reaction (FIG. 7).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method of immobilizing amplicons from a nucleic acid sequence target or fragment thereof, the method comprising:
(i) mixing a fluid sample comprising the nucleic acid sequence target or fragment thereof, magnetic nanoparticles, primers, and an amplification reagent,
wherein the magnetic nanoparticles comprise a first magnetic nanoparticle attached to a first probe and a second magnetic nanoparticle attached to a second probe,
wherein the first probe and the second probe are the same or different and optionally hybridize to different sections of the nucleic acid sequence target or the fragment thereof; and
(ii) heating the fluid sample to activate the amplification reagent and amplify the nucleic acid sequence target or fragment thereof to form amplicons,
thereby immobilizing the amplicons on the magnetic nanoparticles.

2. The method of claim 1, wherein the nucleic acid sequence target or fragment thereof is a deoxyribonucleic acid (DNA) sequence, a ribonucleic acid (RNA) sequence, or a combination thereof.

3. The method of claim 2, wherein the nucleic acid sequence target or fragment thereof is a ribonucleic acid (RNA) sequence and the fluid sample further comprises a reverse transcriptase.

4. The method of claim 1, wherein the mixture is heated at a temperature of at least about 30° C. or at least 50° C.

5. The method of claim 1, wherein the mixture is heated at a temperature between about 60° C. and about 70° C. or between about 60° C. and about 65° C.

6. The method of claim 1, wherein the amplification reagent is a Bst polymerase, a recombinase, a single-stranded DNA-binding protein, a strand-displacing polymerase, an enzyme designed for nucleic acid sequence-based amplification, an enzyme designed for helicase-dependent amplification, a nicking enzyme, or any combinations thereof.

7. The method of claim 1, wherein the amplification reagent yields amplicons with single-stranded regions.

8. The method of claim 7, wherein the single-stranded regions comprise single-stranded loop regions.

9. The method of claim 8, wherein the first probe and the second probe hybridize to the single-stranded loop regions.

10. The method of claim 1, wherein the magnetic material comprises iron, cobalt, nickel, or any combination thereof.

11. The method of claim 10, wherein the iron is an iron oxide with a formula of Fe₃O₄, α-Fe₂O₃, β-Fe₂O₃, FeO, any derivatives thereof, or any combinations thereof.

12. The method of claim 10, wherein the cobalt is a cobalt ferrite with a formula of CoFe, CoFe₂O₄, any derivatives thereof, or any combinations thereof.

13. The method of claim 1, wherein the magnetic nanoparticles further comprise a linker connecting the probe to the surface of the magnetic material, wherein the linker is a functional group, a polymeric group, a dendritic group, or any combinations thereof.

14. The method of claim 13, wherein the linker is a functional group covalently coupling the probe to the surface of the magnetic material.

15. The method of claim 14, wherein the covalent coupling comprises:
a carboxyl-to-amine linkage using 1-ethyl-3-(3-dimethylaminopropyl) carbodiimide and N-hydroxy succinimide (NHS) or sulfo N-hydroxy succinimide;
an amine-to-amine linkage using glutaraldehyde;
an alkyne linkage using an alkyne click chemistry with cyanogen bromide; or
any combinations thereof.

16. The method of claim 13, wherein the linker is a functional group comprising a hydroxyl, a carboxyl, an amine, a mercapto, an epoxy, an imidocarbonate, a cyanate ester, any derivates thereof, or any combinations thereof.

17. The method of claim 13, wherein the linker is a polymeric group comprising poly(ethylene glycol), poly(ethylenimine), poly(thioether), any derivatives thereof, or any combinations thereof.

18. The method of claim 13, wherein the linker is a dendritic group comprising poly(amidoamine), hyperbranched bis-MPA polyester-16-hydroxyl, any derivatives thereof, or any combinations thereof.

19. The method of claim 1, wherein the magnetic nanoparticles have a diameter of at least about 10 nm or at least about 100 nm.

20. The method of claim 1, wherein the magnetic nanoparticles have a diameter between about 100 nm to about 1000 nm.

21. The method of claim 1, wherein the magnetic nanoparticles comprising the immobilized amplicons are agglutinates.

22. The method of claim 1, wherein the magnetic nanoparticles further comprise a shell.

23. The method of claim 22, wherein the thickness of the shell is equal or less than about 5 nm.

24. The method of claim 22, wherein the shell comprises silica embedded with functional groups.

25. The method of claim 24, wherein the functional groups are hydroxyl, carboxyl, amine, mercapto, epoxy, imidocarbonate, cyanate ester, any derivatives thereof, or any combinations thereof.

26. A method for detecting a nucleic acid sequence target or fragment thereof in a fluid sample, the method comprising:
(i) mixing the fluid sample comprising the nucleic acid sequence target or fragment thereof, magnetic nanoparticles, primers, and an amplification reagent,
wherein the magnetic nanoparticles comprise a first magnetic nanoparticle attached to a first probe and a second magnetic nanoparticle attached to a second probe,
wherein the first probe and the second probe are the same or different and optionally hybridize to different sections of the nucleic acid sequence target or the fragment thereof;
(ii) heating the fluid sample to activate the amplification reagent and amplify the nucleic acid sequence target or fragment thereof to form amplicons, thereby agglutinating the magnetic nanoparticles; and
(iii) analyzing the agglutinates to detect the nucleic acid sequence target or fragment thereof.

27. The method of claim 26, further comprising the step of separating the agglutinates from the fluid sample before the analyzing step.

28. The method of claim 27, wherein the separating step comprises filtration, decantation, centrifugation, magnetism, or any combinations thereof.

29. The method of claim 26, wherein the agglutinates are analyzed by visual inspection.

30. The method of claim 26, wherein the agglutinates are analyzed by color intensity measurement, optical density measurement, or a combination thereof.

31. The method of claim 26, wherein the nucleic acid sequence target or fragment thereof is a deoxyribonucleic acid (DNA) sequence, a ribonucleic acid (RNA) sequence, or a combination thereof.

32. The method of claim 26, wherein the nucleic acid sequence target or fragment thereof is a ribonucleic acid (RNA) sequence and the fluid sample further comprises a reverse transcriptase.

33. The method of claim 26, wherein the mixture is heated at a temperature of at least about 30° C. or at least 50° C.

34. The method of claim 26, wherein the mixture is heated at a temperature between about 60° C. and about 70° C. or between about 60° C. and about 65° C.

35. The method of claim 26, wherein in step (i) the magnetic nanoparticles have a magnetic moment of at least about 30 emu/g.

36. The method of claim 26, wherein in step (i) the magnetic nanoparticles have a magnetic moment between about 30 emu/g and about 200 emu/g.

37. The method of claim 26, wherein in step (i) the magnetic nanoparticles are monodispersed.

38. The method of claim 26, wherein the magnetic nanoparticles with the immobilized amplicons have a magnetic moment of at least 100 emu/g.

39. A method of determining the amount of a nucleic acid sequence target or fragment thereof in a fluid sample, the method comprising:
(i) mixing the fluid sample comprising the nucleic acid sequence target or fragment thereof, magnetic nanoparticles, and an amplification reagent,
wherein the plurality of magnetic nanoparticles comprises a first magnetic nanoparticle attached to a first probe and a second magnetic nanoparticle attached to a second probe,
wherein the first probe and the second probe are the same or different and optionally hybridize to different sections of the nucleic acid sequence target or the fragment thereof;
(ii) heating the fluid sample to activate the amplification reagent and amplify the nucleic acid sequence target or fragment thereof to form amplicons, thereby agglutinating the magnetic nanoparticles;
(iii) analyzing the agglutinates to determining the amount of nucleic acid sequence target or fragment thereof in the fluid sample.

40. The method of claim 39, further comprising the step of: (iv) comparing the amount of nucleic acid sequence target or fragment thereof in the fluid sample to a threshold amount or to a control sample with a known amount of nucleic acid sequence target or fragment thereof.

41. A kit for detecting a nucleic acid sequence target or fragment thereof, the kit comprising:
a container suitable for mixing a fluid sample comprising the nucleic acid sequence target or fragment thereof, magnetic nanoparticles, and an amplification reagent, wherein the plurality of magnetic nanoparticles comprises a first magnetic nanoparticle attached to a first probe and a second magnetic nanoparticle attached to a second probe, wherein the first probe and the second probe are the same or different and optionally hybridize to different sections of the nucleic acid sequence target or the fragment thereof; and
a heater capable of heating the fluid sample and activating the amplification reagent to form amplicons, thereby agglutinating the magnetic nanoparticles; and
a means for analyzing the presence of, or the property of, the agglutinates, hereby detecting the nucleic acid sequence target or fragment thereof.

42. An apparatus for detecting a nucleic acid sequence target or fragment thereof using loop-mediated isothermal amplification, the apparatus comprises:
an entry valve for entering a fluid sample comprising the nucleic acid sequence target or fragment thereof, magnetic nanoparticles, and an amplification reagent, wherein the plurality of magnetic nanoparticles comprises a first magnetic nanoparticle attached to a first probe and a second magnetic nanoparticle attached to a second probe, wherein the first probe and the second probe are the same or different and optionally hybridize to different sections of the nucleic acid sequence target or the fragment thereof; and
a plurality of chambers fluidly connected to the entry valve and each other, wherein the chambers are designed to receive the fluid sample, wherein each of the chambers comprises a gel which is embedded with a primer that is specific to the nucleic acid target or fragment thereof;
a heating device to heat the fluid sample, liquify the gel-laden primers in their respective chambers, and activate the amplification reagent to form amplicons by amplifying the nucleic acid sequence target or the fragment thereof using the probes hybridized to the nucleic acid sequence target or fragment, thereby agglutinating the magnetic nanoparticles; and
a magnet to interact with the agglutinates, thereby detecting the nucleic acid target or fragment thereof.
